(19) United States
(12) Patent Application Publication
(10) Pub. No.: US 2007/0155765 A1
(43) Pub. Date: Jul. 5, 2007

(54) Method of preparation of the hemi-calcium salt of (e)-7-[4-(4fluorophenyl)-6-isopropyl-2-[methyl(methylsulfonvl)aminolpyrimidin-5-yl](3r,5s)-3,5-, dihvdroxy-6-heptenoic acid

(75) Inventors: Pavel Sebek, Praha (CZ); Stanislav Radl, Praha (CZ); Jan Stach, Praha (CZ)

Correspondence Address: OBLON, SPIVAK, MCCLELLAND, MAIER & NEUSTADT, P.C., 1940 DUKE STREET, ALEXANDRIA, VA, 22314, US

(73) Assignee: Zentiva, A.S., Praha (CZ)

(21) Appl. No.: 10/585,933

(22) PCT Filed: Dec. 17, 2004
(86) PCT No.: PCT/CZ04/00088

§ 371 Date: Jan. 4, 2007

(30) Foreign Application Priority Data
Jan. 16, 2004 (CZ) PV 2004-86

(51) Int. Cl. A61K 31/505; C07D 239/42

(52) U.S. Cl. 514/275; 544/330

(57) ABSTRACT

A method of preparation of the hemi-calcium salt of rosuvastatin of formula (I) consists in extracting an aqueous solution of the sodium or potassium salt of (E)-7-[4-(4-fluorophenyl)-6isopropyl-2-[methyl(methylsulfonyl)amino]pyrimidin-5-yl](3R,5S)-3,5-dihydroxy-6-heptenoic acid, with optional admixture of sodium or potassium hydroxide or other sodium or potassium salts having inorganic anions, with an organic solvent, incompletely miscible with water, selected from the series of R¹COOR², R¹COR² and R¹OH, wherein R¹ and R² independently represent hydrogen or a residue of a C₁-C₁₀ aliphatic hydrocarbon, C₆ aromatic hydrocarbon, C₅ or C₆cyclic hydrocarbon, or a combination of an aliphatic and aromatic or cyclic hydrocarbon, the extract being subsequently shaken with an aqueous solution of an inorganic or C₁-C₅ organic calcium salt, and the product of formula I is further isolated by cooling and/or adding an anti-solvent and filtration, and optionally, is converted into its amorphous form.

### TECHNICAL FIELD

The invention concerns a new method of preparation of the hemi-calcium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid known under the INN name rosuvastatin, formula I.

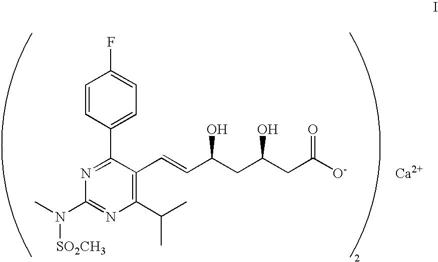

The mentioned medicament is a prominent representative of hypolipidemic and hypocholesteric pharmaceuticals.

### BACKGROUND ART

Rosuvastatin is produced according to the published patent (EP 521471) usually from the sodium salt of (E)-7-[4-(4-fluorophenyl)-6-isopropyl-2-[methyl(methylsulfonyl)amino]-pyrimidin-5-yl](3R,5S)-3,5-dihydroxy-6-heptenoic acid and an appropriate water-soluble calcium salt, preferably from calcium chloride.

The starting sodium salt can be obtained according to the above-mentioned patent from the methyl ester of (E)-7-[4-(4-fluorophenyl)-6-isopropyl-2-[methyl(methylsulfonyl)amino]-pyrimidin-5-yl](3R,5S)-3,5-dihydroxy-6-heptenoic acid of formula II via hydrolysis with ethanolic sodium hydroxide or lately (according to international patent application WO 00/49014) from tert-butyl(E)-(6-[2-[4-(4-fluorophenyl)-6-isopropyl-2-[methyl-(methylsulfonyl)amino]pyrimidin-5-yl]vinyl](4R,6S)-2,2-dimethyl-[1,3]dioxan-4-yl)-acetate of formula III

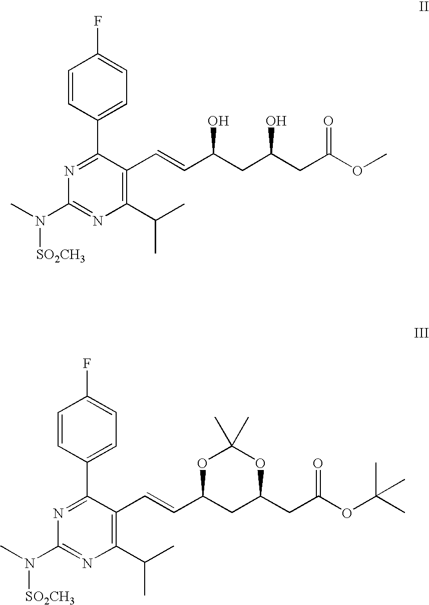

This intermediate product is first transferred to the corresponding sodium salt by consecutive stirring first with hydrochloric acid and then with sodium hydroxide. The calcium salt is subsequently obtained via addition of calcium chloride to the solution of the sodium salt in water. However, the salt prepared in this way is contaminated with inorganic substances. For example, residual sodium hydroxide reacts with calcium chloride to produce water-insoluble calcium hydroxide. Authors of the new patent application (WO 00/042024) assert that the substance prepared according to patent EP 521471 had an amorphous structure; nevertheless the process of its preparation was difficult to reproduce.

According to another patent application (WO 03/016317), the calcium salt can be obtained also via reaction of calcium hydroxide with lactone of formula IV 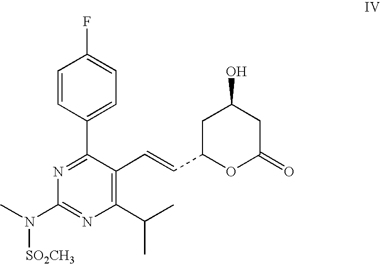
or other esters of rosuvastatin.

The objective of this invention is to describe a new, improved method of preparation of the hemi-calcium salt of (E)-7-[4-(4-fluorophenyl)-6-isopropyl-2-[methyl(methylsulfonyl)-amino]pyrimidin-5-yl](3R,5S)-3,5-dihydroxy-6-heptenoic acid (rosuvastatin), which would not have the mentioned disadvantages, and also an improved method of preparation of the amorphous form.

### DISCLOSURE OF INVENTION

The subject matter of the invention consists in an improved method of preparation of the hemi-calcium salt of (E)-7-[4-(4-fluorophenyl)-6-isopropyl-2-[methyl(methylsulfonyl)-amino]pyrimidin-5-yl](3R,5S)-3,5-dihydroxy-6-heptenoic acid of formula I, wherein an aqueous solution of the sodium or potassium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid, with optional admixture of sodium or potassium hydroxide or other sodium or potassium salts having inorganic anions, is extracted with an organic solvent, incompletely miscible with water, selected from the series of R¹COOR², R¹COR² and R¹OH, wherein R¹ and R² independently represent hydrogen or a residue of a C₁-C₁₀ aliphatic hydrocarbon, C₆ aromatic hydrocarbon, C₅ or C₆ cyclic hydrocarbon, or a combination of an aliphatic and aromatic or cyclic hydrocarbon, the extract being subsequently shaken with an aqueous solution of an inorganic or C₁-C₅ organic calcium salt, and the product of formula I is further isolated by cooling and/or adding an anti-solvent and filtration.

The aqueous solution of the sodium or potassium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid is preferably obtained stepwise by acidic hydrolysis and subsequent alkaline hydrolysis of the protected ester of formula III 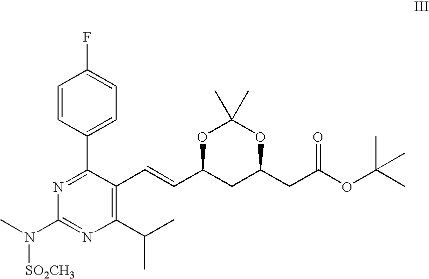
or by alkaline opening of the lactone of formula IV

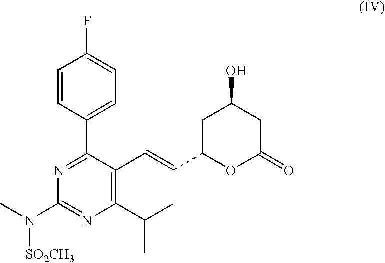

Extraction of the sodium or potassium salt from the aqueous solution is performed with an ester of formula R¹COOR², wherein R¹ and R² have the above mentioned meanings, or, even more preferably, extraction is made with ester R^{1′}COOR^{2′}, wherein R^{1′} and R^{2′} are independently hydrogen or a C₁-C₅ aliphatic residue, preferably with ethyl acetate.

This whole procedure is based on the surprising finding that the sodium or potassium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid can be quantitatively extracted from the aqueous phase into solvents of the type of esters, ketones or alcohols of formulae R¹COOR², R¹COR² or R¹OH, wherein R¹ and R² have the above-mentioned meaning. The sodium or potassium salt obtained in this way can be quantitatively transferred into the calcium salt by stirring with an aqueous solution of an inorganic or organic calcium salt. Rosuvastatin can be subsequently obtained by evaporation and crystallization.

Another aspect of the invention consists in a new method of preparation of the amorphous form, which is based on dissolving the calcium salt of rosuvastatin in a suitable solvent and adding the same to an anti-solvent, in which rosuvastatin is completely insoluble or little soluble. A solution of the hemi-calcium salt of rosuvastatin in an organic solvent selected from the series of R¹COOR², R¹COR² or R¹OH, wherein R¹ and R² have the above-mentioned meaning, is added dropwise to an anti-solvent in which rosuvastatin is insoluble, selected from the series including compounds of formulae R¹H and R¹OR², wherein R¹ and R² have the above-mentioned meaning, or water.

The compound of formula I is dissolved in a solvent preferably selected from the series of R^{1′}COOR^{2′}, R^{1′}COR^{2′} or R^{1′}OH, wherein R^{1′} and R^{2′} have the above-mentioned meanings, added dropwise to an anti-solvent in which rosuvastatin is insoluble, selected from the series including compounds of formulae R^{1′}H, R^{1′OR}^{2′}, wherein R¹′ and R²′ have the above-mentioned meanings, or water.

The compound of formula I is preferably dissolved in a solution including ketones, particularly acetone, ethyl methyl ketone, isopropyl methyl ketone, alcohols, particularly methanol, ethanol, isopropanol, or butanols, and further esters, particularly of formic acid, acetic acid or propionic acid with methyl, ethyl or propyl alcohol, and the product is precipitated with solvents including heptane, pentane, cyclohexane, toluene, petroleum ether, diethyl ether or water.

### BRIEF DESCRIPTION OF DRAWINGS

FIG. 1 shows the diffraction pattern of an amorphous sample of the hemi-calcium salt of rosuvastatin.

### DETAILED DESCRIPTION OF THE INVENTION

Esters of rosuvastatin or rosuvastatin lactone of formula IV can be hydrolyzed in aqueous tetrahydrofuran with sodium hydroxide and the resulting sodium salt of rosuvastatin can be quantitatively extracted into the organic phase, preferably with ethyl acetate. The sodium salt obtained in this way is converted into the calcium salt by shaking a solution of the sodium salt in ethyl acetate or another solvent of the above-mentioned type with a water soluble calcium salt, preferably calcium acetate. The residual inorganic contaminants are subsequently removed by washing with demineralized water. Evaporation and crystallization can produce rosuvastatin, which is not contaminated with inorganic substances.

According to the original patent EP 521471, the prepared rosuvastatin had an amorphous structure, but the process is not reproducible. The amorphous form has usually different dissolution characteristics and bio-availability than crystalline forms (Konno T.: Chem. Pharm. Bull. 1990, 38, 2003). In case of rosuvastatin, which is little soluble in water, it is important to have a reproducible process for obtaining the amorphous form.

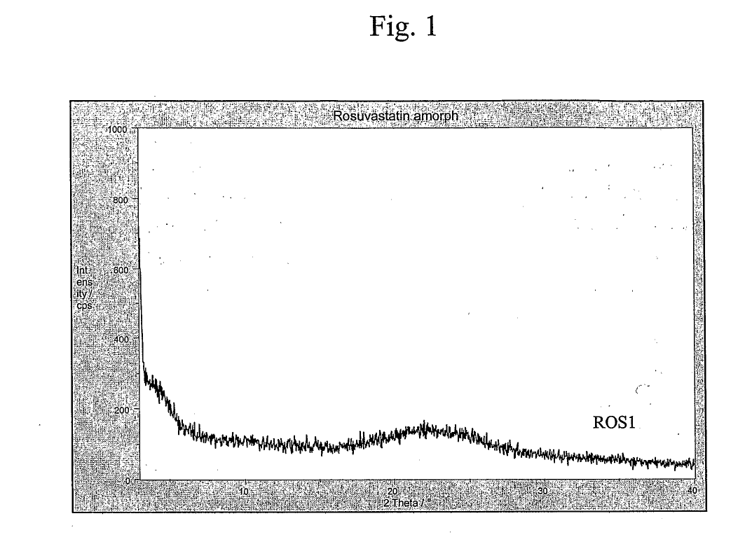

In our method, it has turned out that perfectly amorphous rosuvastatin can be obtained by dissolving crystalline or semi-crystalline rosuvastatin in a solvent in which rosuvastatin is soluble under cold conditions or at increased temperatures, selected from the series of R¹COOR², R¹COR² or R¹OH, wherein R¹ and R² have the above-mentioned meaning, and by adding the resulting solution to an anti-solvent in which rosuvastatin is insoluble, selected from the series of R¹H, R¹OR², wherein R¹ and R² have the above-mentioned meaning, or water. The solvents in which rosuvastatin is soluble under cold conditions or at increased temperatures include those solvents in which solubility is higher than 1 g in 50 ml. Mixtures of suitable solvents can be also used. Examples of such preferable solvents include methanol, ethyl methyl ketone or ethyl acetate. The anti-solvents in which rosuvastatin is insoluble include those in which 1 g of the substance does not dissolve in 1,000 ml of the solvent under cold conditions. Examples of such solvents include preferably hexane, pentane, diethyl ether or water. A more detailed list of these solvents has been presented above. The diffraction pattern of a perfectly amorphous sample (prepared according to Example 5) is shown in FIG. 1; the measurements were performed on diffractometer SEIFERT 3000 XRD with a graphite monochromator, radiation CoKα (λ=1.790 Å) within the range 2.5-40°2θ with a step 0.03.

The invention is elucidated in more detail in the following examples. The examples, which illustrate preferred alternatives of production of rosuvastatin according to the invention, have a purely illustrative character and do not limit the extent of the invention in any respect. Semi-crystalline rosuvastatin used in Example 5 was obtained according to the original patent EP 521471. Crystalline rosuvastatin used in Examples 6 and 7 was obtained according to WO 00/042024.

### EXAMPLES

### Example 1

Tetrahydrofuran (75 ml) is added to lactone IV (5 g, 10.8 mmol). A solution of 40% NaOH (10 ml) is added during 5 minutes to the solution obtained in this way and the formed heterogeneous mixture is vigorously stirred for 17 h and then poured into a separating funnel containing demineralized water (150 ml) and hexane (50 ml). After shaking, the organic layer is separated and the aqueous layer is extracted with a mixture of hexane (40 ml) and tetrahydrofuran (10 ml). After complete separation, the aqueous layer is extracted with ethyl acetate (1×40 ml, 3×20 ml). The ethyl acetate extract is then gradually shaken 3 times with demineralized water (5 ml), each containing 1 g of calcium acetate in 5 ml of water. The resulting ethyl acetate extract is washed with demineralized water (2×5 ml) and, after drying, is concentrated in a vacuum evaporator to a volume of 30 ml and added dropwise to hexane (150 ml) to give, after filtration, 4.5 g of amorphous rosuvastatin.

¹H NMR (DMSO) δ: 1.22 (d, J=7, 6H); 1.41 (m, 1H); 1.61 (m, 1H); 2.18 (dd, J=3, 2H); 3.43 (m, 1H); 3.45 (s, 3H); 3.57 (s, 3H); 3.83 (m, 1H); 4.25(m, 1H); 5.56 (dd, J=7.16, 1H); 6.58 (d, J=16, 1H); 7.33 (m, 2H); 7.76 (m, 2H)

MS for C₂₂H₂₈FN₃O₆SNa [M+Na]⁺: calculated 504.1; found 503.8.

### Example 2

Following the procedure described in Example 1 using potassium hydroxide instead of sodium hydroxide for the hydrolysis of the ester, the corresponding potassium salt of rosuvastatin is obtained. The solution is further treated according to the procedure described in Example 1, to provide 4.2 g of amorphous rosuvastatin.

### Example 3

Tetrahydrofuran (15 ml) is added to ester III (1 g, 1.7 mmol) and after a clear solution is formed, 10% HCl (4 ml) is added. The mixture is stirred for additional 24 hours at ambient temperature. Then, a solution of 40% NaOH (2 ml) is added to the solution during 5 min and the formed heterogeneous mixture is vigorously stirred for 17 h and then poured into a separating funnel containing demineralized water (30 ml) and hexane (10 ml). After shaking, the organic layer is separated and the aqueous layer is extracted with a mixture of hexane (8 ml) and tetrahydrofuran (2 ml). After complete separation, the aqueous layer is extracted with ethyl acetate (1×20 ml, 3×10 ml). Combined ethyl acetate extracts are gradually shaken 3 times with demineralized water (1 ml), each containing 0.2 g of calcium acetate in 1 ml of water. The resulting ethyl acetate solution is washed with demineralized water (2×3 ml) and after drying with calcium sulfate, it is evaporated in a vacuum evaporator. After crystallization from acetonitrile and water, 0.7 g of rosuvastatin is obtained.

### Example 4

Tetrahydrofuran (15 ml) is added to ester II (1 g, 2 mmol) and after complete dissolution, a solution of 40% NaOH (2 ml) is added to the solution over 5 min and the formed heterogeneous mixture is vigorously stirred for 17 h and then poured in a separating funnel containing demineralized water (30 ml) and hexane (10 ml). After shaking, the organic layer is separated and the aqueous layer is extracted with a mixture of hexane (8 ml) and tetrahydrofuran (2 ml). After complete separation, the aqueous layer is extracted with ethyl acetate (1×20 ml, 3×10 ml). The ethyl acetate solution is subsequently shaken 3 times with demineralized water (1 ml), each containing 0.2 g of calcium acetate in 1 ml of water. The resulting ethyl acetate solution is washed with demineralized water (2×3 ml) and evaporated in a vacuum evaporator. After crystallization from acetonitrile and water, 0.7 g of rosuvastatin is obtained.

### Example 5

Semi-crystalline rosuvastatin (1 g) is dissolved in ethyl methyl ketone (10 ml) at 40° C. After being filtered, the resulting solution is added dropwise to pentane (70 ml), while the mixture is vigorously stirred. After 30 min of stirring, the solution is sucked off and dried in vacuo to give 0.95 g of amorphous rosuvastatin.

### Example 6

Crystalline rosuvastatin (1.5 g) is dissolved in methanol (10 ml) at 25° C. After being filtered, the resulting solution is added dropwise to water (150 ml), while the mixture is vigorously stirred at 5° C. After 30 min of stirring, the solution is sucked off and dried in vacuo to give 1.3 g of amorphous rosuvastatin.

### Example 7

Crystalline rosuvastatin (1 g) is dissolved in methanol (10 ml) at 25° C. After being filtered, the resulting solution is added dropwise to diethyl ether (150 ml) at 25° C. After 30 min of stirring, the solution is sucked off and dried in vacuo to give 0.7 g of amorphous rosuvastatin.

1. A method of preparation of the hemi-calcium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid of formula I, i.e. rosuvastatin 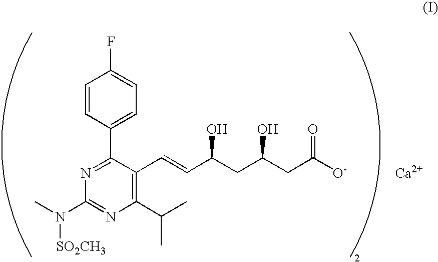
comprising extracting an aqueous solution of the sodium or potassium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid, with optional admixture of sodium or potassium hydroxide or other sodium or potassium salts having inorganic anions, with an organic solvent, incompletely miscible with water, selected from the series of R¹COOR², R¹COR² and R¹OH, wherein R¹ and R² independently represent hydrogen or a residue of a C₁-C₁₀ aliphatic hydrocarbon, C₆ aromatic hydrocarbon, C₅ or C₆ cyclic hydrocarbon, or a combination of an aliphatic and aromatic or cyclic hydrocarbon, subsequently shaking the extract with an aqueous solution of an inorganic or C₁-C₅ organic calcium salt, and further isolating the product of formula I by cooling and/or adding an anti-solvent and filtration, and, optionally, converting the product into its amorphous form.

2. The method according to claim 1 wherein the aqueous solution of the sodium or potassium salt of (E)-7-[4-(4-fluorophenyl)-6-isopropyl-2-[methyl-(methylsulfonyl)amino]pyrimidin-5-yl](3R,5S)-3,5-dihydroxy-6-heptenoic acid is obtained stepwise by acidic hydrolysis and subsequent alkaline hydrolysis of the protected ester of formula III 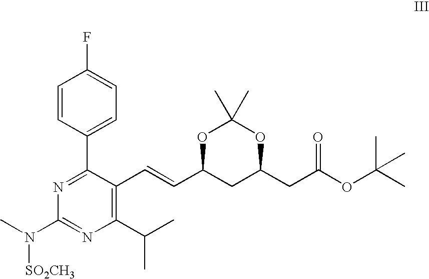
or by alkaline opening of the lactone of formula IV

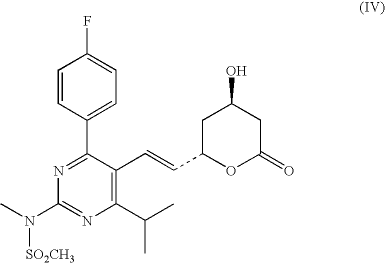

3. The method according to claim 1 wherein the extraction of the sodium or potassium salt from the aqueous solution is performed with an ester of formula R¹COOR², wherein R¹ and R² are as defined in claim 1.

4. The method according to claim 1 wherein the extraction is performed with ester R^{1′}COOR², wherein R^{1′} and R^{2′} are independently hydrogen or a C₁-C₅ aliphatic residue, preferably with ethyl acetate.

5. A method of the preparation of the amorphous form of the hemi-calcium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid of formula I, i.e. rosuvastatin, according to claim 1, wherein a solution of the hemi-calcium salt of (E)-7-[4-(4-fluorophenyl)-6-isopropyl-2-[methyl(methylsulfonyl)amino]pyrimidin-5-yl](3R,5S)-3,5-dihydroxy-6-heptenoic acid in an organic solvent selected from the series of R¹COOR², R¹COR² and R¹OH, wherein R¹ and R² are as defined in claim 1, is added dropwise to a solvent in which rosuvastatin is insoluble, selected from the series including compounds of formulae R¹H and R¹OR², wherein R¹ and R² are as defined in claim 1, and water.

6. The method according to claim 5 wherein the compound of formula I is dissolved in a solvent selected from the series of R^{1′}COOR^{2′}, R^{1′}COR^{2′} and R^{1′}OH, wherein R^{1′} and R^{2′} are independently hydrogen or a C₁-C₅ aliphatic residue, is added dropwise to a solvent in which rosuvastatin is insoluble, selected from the series including compounds of formulae R^{1′}H or R^{1′}OR^{2′}, wherein R^{1′} and R^{2′} are independently hydrogen or a C₁-C₅ aliphatic residue, and water.

7. The method according to claim 5 wherein the compound of formula I is dissolved in a solvent including ketones, particularly acetone, ethyl methyl ketone, isopropyl methyl ketone, alcohols, particularly methanol, ethanol, isopropanol, or butanols, further esters, particularly of formic acid, acetic acid or propionic acid with methyl, ethyl or propyl alcohol, and the product is precipitated with solvents including heptane, pentane, cyclohexane, toluene, petroleum ether, diethyl ether or water.
